(19) United States
(12) Patent Application Publication
(10) Pub. No.: US 2022/0013200 A1
(43) Pub. Date: Jan. 13, 2022

(54) Influenza Status Registry

(71) Applicant: Keith Marz, Sherman Oaks, CA (US)

(72) Inventor: Keith Marz, Sherman Oaks, CA (US)

(21) Appl. No.: 17/347,522

(22) Filed: Jun. 14, 2021

Related U.S. Application Data
(60) Provisional application No. 63/049,700, filed on Jul. 9, 2020.

(51) Int. Cl. G16H 10/60; G16H 10/40; G16H 40/67; G16H 50/80; G06Q 30/00; G06F 8/61

(52) U.S. Cl. CPC G16H 10/60; G16H 10/40; G16H 40/67; G06Q 50/01; G06Q 30/0185; G06F 8/61; G16H 50/80

(57) ABSTRACT

An influenza Status Registry (ISR) that provides a method of acquiring, storing and displaying a person's status regarding influenza, particularly the COVID-19 coronavirus, as well as vaccination information. The ISR uses a software program that operates on a computer or a wireless device such as a smart phone or tablet computer. A person will have an influenza/COVID-19 test performed by a medical professional. The test results, which show the person's status, are then displayed on the computer or wireless device. Any business or other entity where multiple people congregate in a limited space then uses the ISR to determine if it is safe to allow each person to enter the location. The status will be displayed as never had, previously had or currently has influenza/COVID-19, and appropriate action can be taken based on the test results. Additionally, the ISR will display information pertaining to a person's vaccination status.

### TECHNICAL FIELD

The invention generally pertains to medical records, and more particularly to a status registry that provides a method of acquiring, storing and displaying a person's status regarding influenza, particularly COVID-19/coronavirus, as well as vaccination information.

### BACKGROUND ART

Throughout history people have been concerned about their health, and regardless of the cause(s) of health problems such as disease, there has been an attempt to provide a registry. As the practice of medicine became much more understood, and doctors and scientists learned about the human body and sickness/disease/virus, it became apparent that many aspects of human physiology were interconnected. This knowledge resulted in discovery that a cure or procedure meant to treat a medical condition, could have serious effect on many functions of a human body. This was especially noticeable as new drugs or vaccines were developed. A drug or vaccine that offered a solution to a particular disease or virus could also provide to be dangerous.

As a result, it became obvious that it was extremely beneficial (and eventually required) to maintain a record of a person's medical history. By maintaining these records, a medical professional could read about the person and learn all previous medical conditions, procedures, treatments and drugs prescribed. This significantly reduced the number of negative/dangerous drug interactions or if a procedure such as surgery presented more of a risk than an potential benefit. Due to the fact that a comprehensive medical record included information that many people consider private, access to medical records has been strongly controlled.

In our current world of the year 2020 the entire planet is dealing with a pandemic known as COVID-19/coronavirus. As with any other virus that is contracted, a positive result for COVID-19 will be entered into a person's medical record. Unfortunately, as with all information in a medical record, the COVID-19 status is only accessible to medical practitioners or by obtaining a subpoena for the record. Considering the lack of knowledge about COVID-19 and how it spreads throughout the world, it would be extremely beneficial for people's COVID-19 status to be easily and widely accessible. If more people were aware of who actually had COVID-19, proactive steps could be taken to mitigate the human interaction that takes place. Also, if people could possess proof that they did not have COVID-19, businesses and other entities could assure customers and clients that it was safe to interact.

It really would not be feasible to allow access to a selected portion of a person's medical record, but it would be possible to provide a new registry devoted only to influenza and especially COVID-19, as well as vaccination information. It would not be difficult to provide a software based internet-accessed registry that a person could sign-up with to record and display their COVID-19 and vaccination status. A person could retain control of their COVID-19 and vaccination information and, after agreeing to user requirements, could decide who can access their status.

A search of the prior art did not disclose any literature or patents that read directly on the claims of the instant invention. However, the following U.S. patents are considered related:

| Pat. No. | INVENTOR | ISSUED |
|---|---|---|
| 5,361,202 | Doue | Nov. 1, 1994 |
| 5,832,450 | Myers, et al | Nov. 3, 1998 |

The U.S. Pat. No. 5,361,202 patent discloses a computer display system and method for facilitating access to patient data records in a medical information system includes a displayed representation of the duration of the stay of an identified patient in the health care facility. Patient data is stored in data files in a database. Accessed data records may then be displayed and time-stamped.

The U.S. Pat. No. 5,832,450 patent discloses an electronic medical record system that stores data about patient encounters from a content generator. A header for each encounter record stores context information for each encounter record. Each header comprises attributes embodied as a field descriptor and a value, bound together as a text object. By binding the field descriptors to the values, each encounter record is complete in itself, without reference to database keys.

For background purposes and indicative of the art to which the invention relates, reference may be made to the following remaining patents found in the patent search.

| Pat. No. | INVENTOR | ISSUED |
|---|---|---|
| 6,304,848 | Singer | Oct. 16, 2001 |
| 7,490,046 | Wyatt | Feb. 10, 2009 |
| 7,949,544 | Miglietta, et al | May 24, 2011 |
| 8,731,964 | Miglietta, et al | May 20, 2014 |
| 2008/0021739 | Brock | Jan. 24, 2008 |
| 2013/0185087 | Merkin | Jul. 18, 2013 |
| 2014/032449 | Reiner | Oct. 30, 2014 |

### DISCLOSURE OF THE INVENTION

An Influenza Status Registry (ISR) that provides a method of acquiring storing and displaying a person's status regarding influenza, and particularly the COVID-19 coronavirus, as well as vaccination information.

The ISR comprises a software program that operates on a computer or preferably a wireless device such as a smart phone or tablet computer. The software program allows a person to have an influenza/COVID-19 test performed and/or a vaccine administered and the results of the test and vaccination information then displayed when needed. In order for the ISR to be effective, a person will be tested on a regular basis, and the time frame from test to test will be displayed. The testing is performed by a recognized medical professional and the permission to share and display the test results are in compliance with all current medical recording requirements.

The major benefit of the ISR is that businesses, especially those where multiple people are typically present in a small/limited area such as a restaurant/bar, health/beauty services, houses of worship or travel means including airplanes and trains, must be able to determine if a person who has influenza/COVID-19 and/or has been vaccinated is being allowed to interact with other people. While it is acknowledged that there will never be a method that guarantees 100 percent accurate results, the ISR is an effective means of significantly reducing the transmittal of a virus, thereby providing an increased level of safety, and will provide piece of mind to people who must be present at a location with multiple other people. Most people will be much more comfortable entering a location where all persons who are there must show a status if they have, had or never had the COVID-19 virus and/or have been vaccinated. Also, both the virus status and vaccination information will be validated as to authenticity, with all data utilized originating from an established entity such as the Center for Disease Control and Prevention (CDC).

In view of the above disclosure, the primary object of the invention is to produce an Influenza Status Registry that provides a method of acquiring, storing and displaying Influenza/COVID-19 test results as well as validated vaccination information.

In addition to the primary object it is also an object of the invention to produce an Influenza Status Registry that:
is easy to use,
can be used worldwide.
can be used for any business or event where people congregate,
can be kept private,
allows test results to be uploaded automatically, as frequently as required,
complies with all medical legalities,
can save lives, and
is easily implemented by both a user and a provider of services.

These and other objects and advantage of the present invention will become apparent from the subsequent detailed description of the preferred embodiment and the appended claims taken in conjunction with the accompanying drawings.

### BRIEF DESCRIPTION OF THE DRAWINGS

FIG. 1 is a flow diagram showing the steps of an influenza status registry.

### BEST MODE FOR CARRYING OUT THE INVENTION

The best mode for carrying out the invention is presented in terms of a preferred embodiment for a business model for an influenza status and vaccination information registry. For a long time it has been proven to be necessary for a person's medical history to be recorded and available to a person's doctor(s), hospitals (esp. emergency rooms) and other medical practitioners. A person's medical history will typically include any/all medical related information, including past illnesses, medical procedures and diagnoses. Due to the extremely personal nature of the information contained in a medical history, access is limited and in most cases permission must be granted to view a person's medical history.

The influenza status registry (ISR) is not a comprehensive medical history. Rather, the ISR only discloses whether a person has, previously had or has not had an influenza virus as well as information pertaining to vaccine administered. To be even more specific, the ISR is actually focused on the circa 2020 COVID-19/coronavirus strain and the vaccines developed for that strain.

Currently, the most common way to determine a person's COVID-19 status is to just ask them. For obvious reasons this method of accurately determining a person's status can not be relied upon as 100% accurate, therefore causing this method to be extremely unreliable. Even for a person who wants to be completely honest, if that person had the virus but was asymptomatic, there is no way to accurately determine the status.

The ISR 10 offers a solution to this problem by providing a single location which is preferably a password protected, secure website or application (APP), dedicated to acquiring, storing, and displaying a person's test results for influenza, and particularly COVID-19 as well as information pertaining to a person's vaccination status.

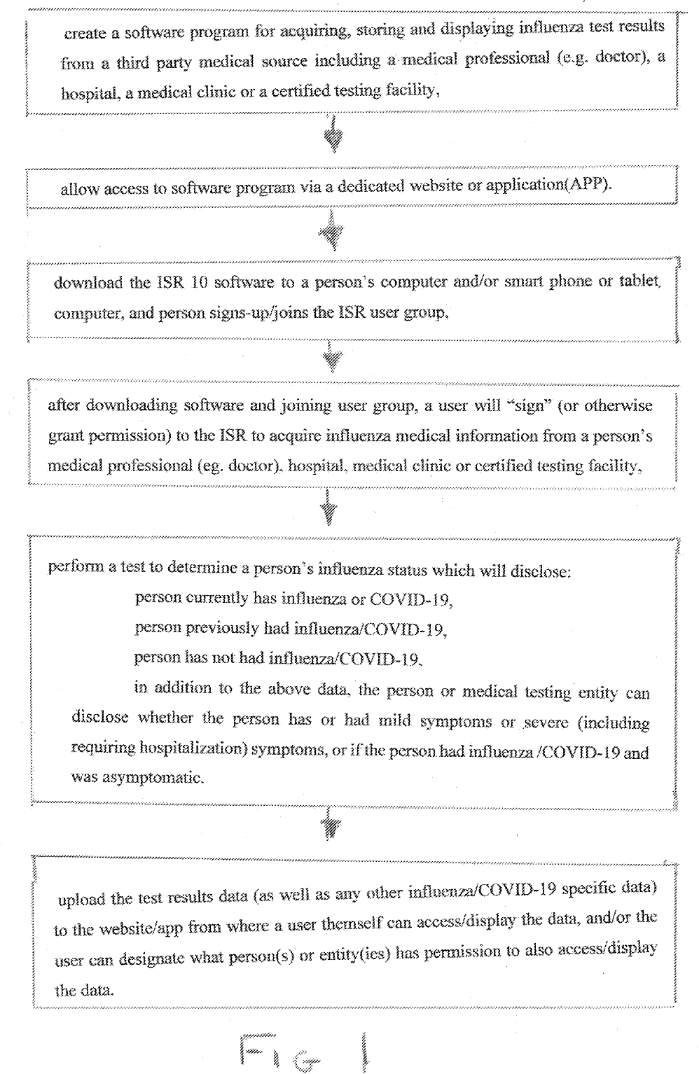

This method by which the ISR 10 functions is accomplished by the following steps as shown in FIG. 1:
1. create a software program for acquiring, storing and displaying influenza test results from a third party medical source including a medical professional (e.g. doctor), a hospital, a medical clinic or a certified testing facility,
2. allow access to software program via a dedicated website or application (APP), download the ISR 10 software to a person's computer and/or smart phone or tablet computer, and person signs-up/joins the ISR user group,
3. after downloading software and joining user group, a user will “sign” (or otherwise grant permission) to the ISR to acquire influenza medical information from a person's medical professional (eg. doctor), hospital, medical clinic or certified testing facility,
4. have a test performed to determine a person's influenza status which will disclose: person currently has influenza or COVID-19,
   person previously had influenza/COVID-19,
   person has not had influenza,
   in addition to the above data, the person or medical testing entity can disclose whether the person has or had mild symptoms or severe (including requiring hospitalization) symptoms, or if the person had influenza/COVID-19 and was asymptomatic.
5. the above data (as well as any other influenza/COVID-19 specific data) is uploaded to the website/app from where a user themself can access/display the data, and/or the user can designate what person(s) or entity(ies) has permission to also access/display the data.

It is important to note that the permission granted by a person for another person(s) and/or entity(ies) to access the person's influenza/COVID-19 data or vaccination information on the website/APP is done in complete legal compliance with the Health Insurance Portability and Accountability Act of 1996 (HIPAA) as well as other medical records protection protocols including Patient Care Management Systems (PCMS) and Electronic Medical Records (EMR). The granting of release of influenza/COVID-19 medical data or vaccination information is limited only to that which pertains to influenza/COVID-19. The release removes any/all non-related liability to a medical professional (eg. doctor), hospital, medical clinic, or certified testing facility, and liability to any person who is given permission to access/display the influenza/COVID-19 or vaccination information.

Also, access to a person's ISR 10 status can be selectively limited. For example, a person could allow all access, thereby permitting any person or group to access a person's ISR status, or only specially designated and identifiable individuals can access a person's ISR status. Another option would be to allow business entities only (either a selected business or any business) to access the ISR status. These options are chosen by person when they sign up for the ISR 10, and the choice(s) can be modified at any time.

The test results displayed on the ISR website/APP are used as a proof of condition. A person is provided with a physical medium such as a document or a card on which their influenza/COVID-19 status is disclosed, or a virtual display is displayed on a person's computer, smart phone or tablet computer. These documents, cards or displays do not include comprehensive data related to the person. Rather, a simple, succinct statement such, as “‘person's name’ has not contracted COVID-19 and now has antibodies”, or “‘person's name’ currently has COVID-19”.

It should be noted that the ISR software can allow a person's status to be modified. This allows a person who currently has COVID-19 to wait a period of time and then when they recover can be re-tested and the status changed depending on the test results. A person who is negative one day could become positive the next day. If a medical professional, hospital, clinic or certified testing facility chooses (or in an alternate scenario is mandated) to become affiliated with the ISR 10, and they upload a person's test results, they can also have as an obligation to update a person's status if/when the person's status changes, either positively or negatively. In lieu of this. a person would be responsible for updating/modifying their status if/when they become aware the status has changed. The update/modifying will occur within a selected amount of time, preferably only hours. A person who signs up for and uses the ISR10, will be required to agree to terms of service (TOS) conditions. as well as sign a disclaimer that he/she will not use the ISR for any illegal purpose or to gain an advantage using known false data. The person will also agree if they are uploading their own test results, they will update/modify their status as soon as they become aware of a change in the status. Optimally, this requirement will have legal repercussions if not adhered to.

In an alternate embodiment, a person who tests positive for COVID-19 does not proceed with any action since they will most likely (per statistical results) recover within a one to two week time period. Displaying test results will usually not be necessary (because the person is recovering and has no need to show that they are ill), or in worst-care scenarios such as long-term hospitalization or death, the ISR website/APP becomes superfluous/redundant.

Additionally, while the ISR is effective for use as a COVID-19 positive or negative registry, the ISR 10 can also be used for a person's vaccination status. Now that vaccine(s) have been developed, it is planned for a majority of the world's population to be vaccinated. The ISR can incorporate a vaccination status along with the COVID-19 status, or a separate registry utilizing the ISR COVID-19 design parameters, can be implemented. The vaccination information can include the type of vaccination (ie, what is the person being vaccinated against); the specific drug used to vaccinate (and optionally, the manufacturer of the drug); the number of vaccinations administered, for example, a first vaccination or a second, follow-up vaccination; the date of each vaccination and/or the location where a vaccination was administered. Also, in a simplified embodiment, the vaccination information will consist of just a yes or no answer to the question of whether a person has been vaccinated. The combined information of a person's COVID-19 status and their vaccination status would significantly increase the efficacy of the ISR 10. In order to provide a strong clarity of status, for both the COVID-19 status and vaccination status, a proof medium, similar to a “passport” could be utilized. This would allow an individual or business to quickly and easily view comprehensive information for any person. An additional component of the ISR vaccination information is a means by which the authenticity of the vaccination information is validated. This would allow people to be assured that the vaccination information originates from a reliable/established entity (such as the U.S. Center for Disease Control and Prevention—CDC) and is valid to the greatest extent possible. A system such as this would be particularly effective for a situation such as a business where all of the employees are on the ISR 10. In this manner, the ISR would function as a single, one-stop method of determining/proving a person's COVID-19 status and vaccination status.

It was been established that there are other entities attempting to implement a registry of virus or vaccination status. One such entity is the U.S. Center for Disease Control (CDC), who have publicly disclosed how they are implementing their status system. As would be expected, unscrupulous individuals are producing and selling forged/fake status documentation, particularly vaccination information. The only official proof of vaccination today is from the CDC, therefore there needs to be protection from the forged/fake documentation. The ISR 10 addresses this problem by providing information on a person's influenza and/or vaccination status as well as a provable, nonduplicatable means that a person has been vaccinated and to validate the authenticity and accuracy of a certification from the CDC. Depending on how a person's status information is displayed (either a document, a card or via a software based display on a mobile device) various types of validating means could be utilized, including a scannable code such as a QR code or barcode; an embedded strip (for physical media) or a displayed strip on a screen; a series of numbers or letters that are verifiable; a tamper-proof enclosure (for physical media) or an indication if information in software has been accessed or modified. It should be noted that these are only examples and there are other current-technology based anti-fraud devices/means that could be used. It is difficult to under emphasis the importance of being able to validate information disseminated from an agency such as the CDC. If them is no way of distinguishing genuine documentation from forged/fake documentation, the production and purpose of the documentation becomes meaningless.

Since the ISR 10 is a business model designed to produce revenue there are multiple methods to accomplish this. In order to join and use ISR 10 a person could be required to pay either a one-time joining cost, or time based (such as monthly, bi-yearly, annually) participation fees could be charged. Users could also be allowed to join and use the ISR 10 for fee, and revenue could be produced by advertising on the website/APP. If the ISR 10 does become popular and has a large number of users, the revenue from advertising will be extremely effective for revenue production.

The ISR 10 can also preferably acquire a service mark and/or domain name that is unique and easily remembered. The servicemark/domain name can then be placed on apparel such as hats. T-shirts, etc. or the service mark can be used as an identifier and phrases such as “I am. COVID-19 free” or “I had COVID-19 but now have the antibodies” can be displayed on an apparel item, along with the ISR service mark/domain name which acts as a bona fide for the statement.

Additionally, one of the most important functional aspects of the ISR 10 is the integration with social media. It is established that a business can utilize the ISR 10 as a verification means to determine who is allowed to either the business or who will receive a service the business provides. For example, to enter a restaurant or bar, to attend a sporting or entertainment event, or to have a serviceperson come to your house for a repair, a person would need to show their ISR status document/card.

For use with social media the same principle applies. A person could simply want to let the (social media) world know their COVID-19 status, so the ISR website/APP could interface with a site such as Facebook and the person's status would appear on the person's Facebook homepage with the ISR servicemark/domain name to provide legitimacy and a link to the ISR website/APP and/or another entity such as the CDC. For other sites, particularly dating sites, a person's ISR 10 status could potentially be much more important. It would obviously be extremely beneficial, and could provide significant peace of mind. For a person searching for any type of relationship to be able to know what can be proven factual regarding a person's COVID-19 status and/or vaccination status. While it is hopeful that most people would disclose their COVID-19 status. or just not participate in on-line dating or relationships, if they are positive. Unfortunately, there are cases where a person has COVID-19 and is asymptomatic, or a person is selfish and does not care about other people.

Additionally, the ISR 10 website/APP will provide various information pertaining to influenza/COVID-19, including confirmed cases by region, as well as information about any prophylactic medication, vaccine or other cure/treatment that becomes available. Any ISR 10 registered person will have a note on their status disclosing any medicine/vaccine (prescription or non-prescription) or treatment the person is taking or undergoing. There will also be an option for a person's ISR status to display relatives, friends or associates who also contracted influenza/COVID-19. A person will also have limited space to describe the symptoms they experienced if they tested positive.

It is further envisioned that in the future, depending on how COVID-19 progresses, the ISR website/APP could contract with medical professionals. hospitals, medical clinics, or certified testing facilities to provide remote video diagnosis, thereby allowing a person who signs up on the ISR 10 to have regular medical assessments performed without having to leave their home or office. A person could take their own temperature, report how they am feeling, report any/all problems and, most importantly, a medical practitioner can visually observe a person and see the person's physical condition. Also, the effectiveness of a vaccine can be accurately monitored, giving medical professionals the ability to modify vaccine protocols if necessary. From all of this a person's status could (in most cases) be accurately determined.

Regardless of how the ISR 10 functionality is implemented and used. and how revenue is (or is not) gained, there is a genuine need for accomplishing what the ISR 10 does. The future is uncertain and the efficacy of the now available vaccine to stop COVID-19 is not guaranteed. Therefore, having the ability to accurately determine and display a person's COVID-19 status and/or vaccination status can only be positive. Even with a vaccine that is widely available, the ISR 10 could result in a significant number of lives potentially being saved, and at the least, people being able to worry less above going out and interacting with others. Also, the ISR 10 can be expanded or modified to include other diseases/viruses or vaccines, including those currently known, as well as those un-discovered as of now. For example, if the COVID-19 virus mutates into a new virus, the ISR can also be utilized for the new virus. The ISR 10 can either have a new virus added along with the current influenza/COVID-19, or a separate ISR can be provided for the new virus and associated vaccination(s).

As new vaccines are developed, the ISR 10 can be updated and include information pertaining to the new vaccines. Also, as forged/fake documentation, particularly pertaining to vaccination information from the CDC, is rampant, the ISR 10 can provide means of validating any/all information from the CDC.

Also, there has currently been a major increase in the number of person's attempting to enter/migrate both legally and illegally to the United States. A significant percent of these persons, or migrants, are unaccompanied minors. It has been estimated that approximately sixty percent of the migrants have the COVID-19 virus. Exacerbating the situation is that many of the migrants are released to various locations throughout the United States. The inclusion of the ISR/COVID Safe List as part of a person's immigration/asylum documentation would allow the U.S. Government or other associated entity to maintain a knowledge base of who among the migrants has COVID-19, who has been vaccinated and where they are located. It would not be difficult to include the ISR/COVID Safe List into a person's documentation. This would provide a migrant's name, gender. age, county of origin, date of entry into the United States and location where they are residing. Having quick access to this information could result in the Government as well as other entities the ability to effectively manage the spread of COVID-19.

In conclusion, it should be known that there are positive and negative aspects to both a person's COVID-19 status and their vaccination status. At this point, the most significant aspect is that people are concerned about safety. In order to feel safe a person needs to have some type of assurance that people they interact with previously had the COVID-19 virus and/or have been vaccinated. The most effective way to accomplish this would be to provide a form of virus and/or vaccination status verification, with established accuracy. The only truly accurate status verification would have to originate form an entity that has obtained and compiled actual validated status verification from either people receiving a vaccination or administering vaccinations. It is the purpose of this Business Model to disclose Intellectual Property that covers the development of what is known as a passport, database comp list, registry or any other name used. Also, it is implied in the spirit of the invention that there are numerous technological means to provide verification, including but not limited to, Quick Response (QR) code, Universal Product Code (Barcode), Identification cards (with or without pictures) or pictures/printed copies of actual vaccination cards. It is important to state that the level of accuracy of, for example, a passport model will be contingent upon access to and assistance/cooperation from the CDC, who are currently the only entity possessing verifiable access to the required data.

One of the major ramifications of the ISR Business Model Intellectual Property is that if a patent is granted, any entity relying on data from the CDC in order to provide some type of passport (or other verification means) would be infringing on both the actuality of and spirit of the patent. This is an important distinction: there is no implication whether the CDC is free to use their proprietary database(s) however they deem fit. Rather, the focus is more on third-parties who are accessing and using the CDC's data. With a granted patent, it would appear to be most logical to encourage the CDC to assist/cooperate with the patent holder to determine which entity will be able to provide the level of safety necessary to those requiring or desiring such safety. The basic fact is that any third-party using the data without the patent holder's permission would be infringing on the patent. This also applies to any entity with personal or business reasons who is interested in the ability to provide vaccination status verification.

Another issue is that there are certain individuals who object to any type of passport/database that acquires and stores information pertaining to vaccination status. It is not the intention of the ISR business model to compromise any privacy issue, and therefore one embodiment of the ISR simply requires a yes or no answer to whether a person has been vaccinated. While some people feel the disclosing of any “medical” related information is an invasion of privacy, it should be noted that everyone has a right not to be exposed to a deadly virus. Especially if the means to prevent exposure are available.

And finally, as previously disclosed, the production and distribution of forged/fake vaccination cards has become extremely pervasive. A great deal of controversy and polarization has developed around questions/issues of determining what/which vaccination card(s) are legitimate. Again, an entity that would be able to determine whether an individual has actually received vaccine would have to either be the CDC or have access to the CDC's database (and record keeping apparatus). Hopefully, a third-party (with permission from the patent holder if applicable) would be able to gain the necessary access to provide a true and accurate yes or no answer to any government, state, airline, train, cruise ship, theme park, theatre and many others.

While the invention has been described in detail and pictorially shown in the accompanying drawings it is not to be limited to such details, since many changes and modifications may be made to the invention without departing from the spirit and the scope thereof. Hence, it is described to cover any and all modifications and forms which may come within the language and scope of the claims.

1. An Influenza Status Registry (ISR) comprising the following steps:
create a software program pertaining to influenza test results,
download the software program to a computer or wireless device,
perform a test to determine a person's influenza status,
upload test results to the ISR software program, and
display test results to a selected entity.

2. The Influenza Status Registry of claim 1, wherein the influenza is comprised of COVID-19 coronavirus.

3. The Influenza Status Registry of claim 1, wherein the person's influenza status is selected from the group consisting of person previously had influenza, person has not had influenza, and person currently has influenza.

4. The Influenza Status Registry of claim 4, wherein the influenza status disclosing a person previously had influenza or person currently has influenza further comprises information disclosing type of symptoms a person experience or is experiencing, or if a person is asymptomatic.

5. The Influenza Status Registry of claim 1, wherein the uploaded test results that are displayed are in legal compliance with the Health Insurance Portability and Accountability Act of 1996 (HIPAA), Patient Care Management Systems (PCMS) and Electronic Medical Records (EMR).

6. The Influenza Status Registry of claim 1, wherein the ISR status is updated or modified as a person has subsequent tests performed.

7. The Influenza Status Registry of claim 1, further comprising proof of vaccination information.

8. The Influenza Status Registry of claim 7, further comprising means for validating the authenticity and accuracy of a certified influenza or vaccination status from a third party.

9. The Influenza Status Registry (ISR) comprising the following steps:
create a software program for acquiring, storing and displaying influenza test results,
access the software program via a dedicated website or computer application (APP),
download the software program to a computer or wireless device,
a person signs up or joins an ISR user group and creates a personal status page, allow ISR user group to acquire medical information from a user's medical professional,
perform a test to determine a user's influenza status and upload test results to the user's personal status page, and
display test results to a selected entity, allowing influenza status to be confirmed by a third party.

10. The Influenza Status Registry of claim 9, wherein the influenza is comprised of COVID-19 Coronavirus.

11. The Influenza Status Registry of claim 9, wherein the wireless device is comprised of a smart phone or tablet computer.

12. The Influenza Status Registry of claim 9, wherein the person's influenza status is selected from the group consisting of person previously had influenza, person has not had influenza, and person currently has influenza.

13. The Influenza Status Registry of claim 12, wherein the influenza status disclosing a person previously had influenza or person currently has influenza further comprises information disclosing type of symptoms a person experienced or is experiencing, or if a person is asymptomatic.

14. The Influenza Status Registry of claim 9, wherein the ISR test results are valid for a selected amount of time.

15. The Influenza Status Registry of claim 9, wherein the uploaded test results that are displayed are in legal compliance with the Health Insurance Portability and Accountability Act of 1996 (HIPAA), Patient Care Management Systems (PCMS) and Electronic Medical Records (EMR).

16. The Influenza Status Registry of claim 9, wherein the ISR status is updated or modified as a person has subsequent tests performed.

17. The Influenza Status Registry of claim 9, wherein the ISR status is an influenza proof of condition displayed on the computer or wireless device, or via physical media configured as a document or card.

18. The Influenza Status Registry of claim 9, further comprising proof of vaccination information.

19. The Influenza Status Registry of claim 18, wherein the proof of vaccination information is comprised of a yes or no answer to a question disclosing whether a person has been vaccinated.

20. The Influenza Status Registry of claim 18, wherein the vaccination information is selected from the group consisting of type of vaccination, drug used to vaccinate, number of vaccinations administered, date of each vaccination and location where vaccination was administered.

21. The Influenza Status Registry of claim 18, wherein the vaccination information is disclosed singularly, or in combination with the influenza status information.

22. The Influenza Status Registry of claim 9, further comprising means for validating the authenticity and accuracy of a certified influenza or vaccination status from a third party.

23. The Influenza Status Registry of claim 22, wherein the validation means is selected from the group consisting of a scannable code, an embedded strip, a displayed strip, a series of numbers or letters that are verified, a tamper-proof enclosure, and an indication if software-based information has been accessed or modified.

24. The Influenza Status Registry of claim 22, wherein the third party is selected from the group consisting of a government agency, an entity affiliated with or working in combination with a government agency and a non-government entity.

25. The Influenza Status Registry of claim 9, wherein the ISR further comprises information that is a required component of a person's immigration or asylum documentation, wherein the ISR information is segregated by a person's gender, age, county of origin and date of entry into the United States.
